SEQUENCE LISTING (18 sequences)

SEQ ID NO: 1 - protein, 21 aa, Artificial Sequence
  Description of Artificial Sequence Synthetic peptide
KHHHKHHHKHHHKHHHKCSSC

SEQ ID NO: 2 - protein, 5 aa, Artificial Sequence
  Description of Artificial Sequence Synthetic peptide
APRPG

SEQ ID NO: 3 - protein, 5 aa, Artificial Sequence
  Description of Artificial Sequence Synthetic peptide
CGKRK

SEQ ID NO: 4 - protein, 7 aa, Artificial Sequence
  Description of Artificial Sequence Synthetic peptide
HAIYPRH

SEQ ID NO: 5 - protein, 8 aa, Artificial Sequence
  Description of Artificial Sequence Synthetic peptide
GPLGIAGQ

SEQ ID NO: 6 - protein, 7 aa, Artificial Sequence
  Description of Artificial Sequence Synthetic peptide
VHLGYAT

SEQ ID NO: 7 - protein, 20 aa, Artificial Sequence
  Description of Artificial Sequence Synthetic peptide
YTRDLVKDPARPKIQKTCTF

SEQ ID NO: 8 - protein, 10 aa, Artificial Sequence
  Description of Artificial Sequence Synthetic peptide
  MOD_RES (6)..(7): D-amino acid
QHTSYKCLRP

SEQ ID NO: 9 - protein, 12 aa, Artificial Sequence
  Description of Artificial Sequence Synthetic peptide
CGGNHWAVGHLM

SEQ ID NO: 10 - protein, 28 aa, Artificial Sequence
  Description of Artificial Sequence Synthetic peptide
YTWMPENPRPGTPCDIFTNSRGKRASNG

SEQ ID NO: 11 - protein, 21 aa, Artificial Sequence
  Description of Artificial Sequence Synthetic peptide
KHHHKHHHKHHHKHHHKSSSC

SEQ ID NO: 12 - protein, 21 aa, Artificial Sequence
  Description of Artificial Sequence Synthetic peptide
KHHHKHHHKHHHKHHHKCSSC

SEQ ID NO: 13 - protein, 20 aa, Artificial Sequence
  Description of Artificial Sequence Synthetic peptide
KHHHKHHHKHHHKHHHKSSC

SEQ ID NO: 14 - protein, 17 aa, Artificial Sequence
  Description of Artificial Sequence Synthetic peptide
KHHHKHHHKHHHKHHHK

SEQ ID NO: 15 - protein, 19 aa, Artificial Sequence
  Description of Artificial Sequence Synthetic peptide
  MISC_FEATURE (9)..(9): May or may not be present
KHHHKHHHHKHHHKHHHKC

SEQ ID NO: 16 - protein, 19 aa, Artificial Sequence
  Description of Artificial Sequence Synthetic peptide
  MISC_FEATURE (9)..(9): May or may not be present
KHHHKHHHHKHHHKHHHKC

SEQ ID NO: 17 - protein, 20 aa, Artificial Sequence
  Description of Artificial Sequence Synthetic peptide
  MOD_RES (18)..(18): Cys or Ser
KHHHKHHHKHHHKHHHKXSS

SEQ ID NO: 18 - protein, 18 aa, Artificial Sequence
  Description of Artificial Sequence Synthetic peptide
KHHHKHHHHKHHHKHHHK

(19) United States
(12) Patent Application Publication
(10) Pub. No.: US 2022/0331441 A1
(43) Pub. Date: Oct. 20, 2022

(54) Tumor-Targeting Polypeptide Nanoparticle Delivery System for Nucleic Acid Therapeutics

(71) Applicant: Sirnaomics, Inc., Gaithersburg, MD (US)

(72) Inventors: Xiaoyong LU, Gaithersburg, MD (US); Patrick Y. LU, Gaithersburg, MD (US); David M. EVANS, Gaithersburg, MD (US)

(21) Appl. No.: 17/713,037

(22) Filed: Apr. 4, 2022

Related U.S. Application Data
(63) Continuation of application No. PCT/US2020/054251, filed on Oct. 5, 2020.
(60) Provisional application No. 62/910,760, filed on Oct. 4, 2019.
(60) Provisional application No. 62/915,450, filed on Oct. 15, 2019.

(51) Int. Cl. A61K 47/64; A61K 47/54; A61K 47/55; C12N 15/113; C12N 15/115

(52) U.S. Cl. CPC A61K 47/641; A61K 47/545; A61K 47/55; C12N 15/113; C12N 15/115; C12N 2310/14; C12N 2310/141; C12N 2310/16; C12N 2310/127

(57) ABSTRACT

A novel nucleic acid delivery system is provided containing a linear histidine-lysine rich cysteine-containing peptide bearing a targeting function, and a four branched histidine-lysine rich polypeptide. The delivery system includes a nucleic acid, such as an siRNA. The components form a nanoparticle complex through multiple non-covalent interactions between the phosphates of siRNA and histidine/lysine of the polypeptide, with reduced toxicity. The stable complex selectively delivers the genetic material to cells. The targeting function enhances the efficiency of the nucleic acid delivery and transfection.

Carrier molecules also are provided that have the ability to deliver a therapeutic molecule to a specific cell within a tissue in the body. The carrier molecule is modified with a targeting ligand capable of binding to specific receptors present or upregulated on the cell to be targeted. The therapeutic molecule is an siRNA, miRNA, or other oligonucleotide. The targeting moiety is a small molecule, peptide, or protein that shows an affinity for a receptor present on the cell to be targeted.

### CROSS REFERENCE TO RELATED APPLICATIONS

This application is a continuation of International Application No. PCT/US2020/54251, filed Oct. 5, 2020, which claims priority under 35 U.S.C. § 119(e) from U.S. Provisional Patent Application Ser. No. 62/910,760, filed Oct. 4, 2019, and U.S. Provisional Patent Application Ser. No. 62/915,450, filed Oct. 15, 2019, each of which are hereby incorporated by reference.

### SEQUENCE LISTING

The instant application contains a Sequence Listing, which has been submitted electronically in ASCII format and is hereby incorporated by reference in its entirety. Said ASCII copy, created on Dec. 11, 2020, is named 4690_0026i_SL.txt and is 5,732 bytes in size.

### FIELD OF THE INVENTION

Delivery systems for nucleic acids and methods of use are provided, including methods for targeted delivery or local delivery of nucleic acid molecules.

### BACKGROUND OF THE INVENTION

Targeted delivery of therapeutics has attracted great interest and benefit to improve tumor treatment through increasing efficacy and reduced side effects. It is believed that accumulation of the nanoparticles (NPs) in tumors is by enhanced permeability and retention (EPR) effect (Maeda, Bioconjugate Chemistry, 21:797-802 (2010)). Thus, the tumor delivery can be improved by coating the particle with tumor-localizing ligands. The mechanism by which ligands increase the antitumor efficacy of their cargo (such as siRNA) is still under debate. Enhanced binding to the tumor surface marker can increase accumulation of NPs in the tumor compared to that of nontargeted tissue. Other investigators have claimed that accumulation of targeted and nontargeted NPs within tumor cells was comparable. It was suggested that increased efficacy of the targeted NPs was caused by the enhanced receptor-mediated endocytosis and increased intracellular localization of siRNA therapeutic. Bartlett et al., (2007): Proc. Nat'l Acad. Sci. USA, 104:15549-15554 (2007). Most possibly, both of the mechanisms have played a vital role in the ligand-targeted therapy and efficacy.

Targeted delivery of siRNA in vivo has been challenging due to their degradation by serum nucleases and rapid clearance, endosomal entrapment, and innate immunity simulation by the nanoparticles (NPs). Recently, a very limited method has been developed in preclinical and clinical trials for the targeted delivery of siRNA. One approach is by Alnylam. It has developed GalNAc-siRNA conjugates, in which a synthetic triantennary N-acetylgalactosamine-based ligand (GaLNAc) is conjugated to chemically modified siRNA. This has enabled efficient, ASGPR-mediated delivery to hepatocytes. Maja et al.; Nature Communications, 9:723 (2018). GaLNAc targets the hepatocyte-specific asialoglycoprotein receptor (ASGPR) in liver. One of the examples is Fitusiran (ALN-AT3, phase II clinical trial, Alnylam) for the treatment of hemophilia and rare bleeding disorders (RBDs) by Sanofi Genzyme. It is subcutaneously administered and the RNAi therapeutic aims to target antithrombin (AT). In another case, the targeting ligand has been incorporated into a liposome formulation when multiple components have been co-assembled together with the siRNA. This type of system retains many of the challenges in terms of the stability of the liposome, biocompatibility, toxicity, production and long term storage in large scale. Leng et al., J. Drug Delivery, ID6971297, (2017). Most recently, nanoparticles formed by the polypeptide/polymer and siRNA have efficiently delivered the siRNA in vivo and some of these products have entered into early clinical trials. For example, a histidine (H)-lysine (K) rich polypeptide has safely and effectively delivered the dual siRNA to its target to achieve therapeutic efficacy. One of the leading drugs is being studied in a clinical phase IIa trial. See: Zhou et al., Oncotarget, 8:80651-80665 (2017); WO2011/140285.

### BRIEF DESCRIPTION OF THE FIGURES

FIG. 1. Tumor Targeting HKC/HKP or HKP(+H) Polypeptide Nanoparticle Delivery System. The graph shows that the formation of a tumor targeting polypeptide nanoparticle between (A) a branched polypeptide H3K4B (HKP) or H3K(+H)4b or (HKP(+H)) with specific histidine/lysine sequence, (B) a linear polypeptide functionalized through a terminal cysteine with a tumor targeting ligand such as RGD, folate, or SmAb, etc. and selected siRNA, and its HKC polypeptide-siRNA nanoplex formation.

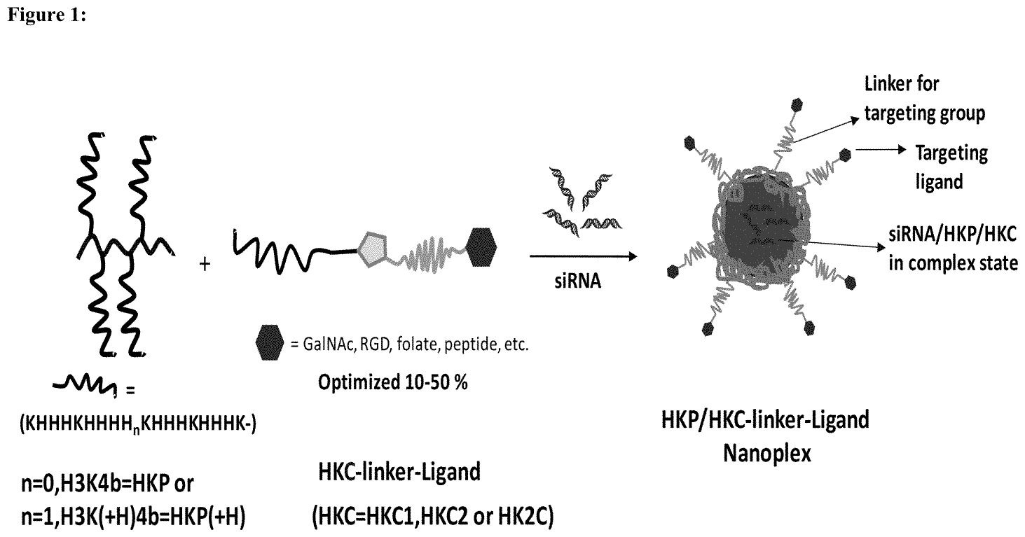

FIG. 2. General scheme of preparation of the HKC-PEG-Targeting ligand functionalized polypeptide (HKC=HKC1, HKC2 or HK2C see FIG. 3). HKC bears a terminal cysteine was conjugated with a Maleimide functionalized PEG linked targeting motif such as (folate, RGD, mAb, etc.) through thiol/maleimide addition reaction under a mild condition.

FIG. 3. Structure of H3K4b (HKP in abbreviation) branched peptide, structure of the H3K4C (in abbreviation HKC1 or HKC) with one cysteine at the terminal site, and structure of HKC2 with two cysteine in the sequence. Two branched cysteine containing peptide HK2C with a sequence [(KHHH)₄]₂KXC. FIG. 3(b) discloses SEQ ID NO: 15 and FIG. 3(c) discloses SEQ ID NO: 16.

FIG. 3B. The HPLC chromatogram of HKC, reverse phase Alltima TM column C-18 (4.6×250 mm), elute at RT=15.196, >91% by the gradient of water (0.065% TFA) and acetonitrile (0.05% TFA).

FIG. 3C. Mass spectroscopy (ESI-MS, positive) of the HKC1 compound, observed double charged molecular ion peak at 1335.6 [M]²⁺.

FIG. 4. Shows the preparation route of HKC2-Peg₁₀₀₀-folate, HKC1 was reacted with Maleimide-PEG-folate through the thiol/maleimide addition reaction under basic condition to provide the conjugation product. After remove the solvent then purification by dialysis, the HKC1-Peg₁₀₀₀-folate was obtained.

FIG. 5. Characterization of HKC2-PEG1k-folate by ¹H NMR, (top) HKC2 in D₂O, and (middle) HKC2-PEG1k-folate in DMSO-d₆, and (bottom) folate-PEG1k-Mal. The HKC was covalently coupled with folate-PEG1k-Mal, the characteristic signal of maleimide double bond at 7.0 ppm was disappeared after reacted with cysteine.

FIG. 6. Shows the UV/Vis (water, 25° C.) spectroscopy of HKC2-PEG1k-folate (top red curve) and Folate-PEG1k-Mal (bottom gray curve) in water. The characteristic absorbance at the 220 nm for the peptide and 275 nm for the folate was observed in the product spectrum.

FIG. 7. Shows the MALDI-MS (positive) spectroscopy of the HKC2-PEG1k-folate, the molecular ion peak around 4302 M⁺ indicates that the successful conversion of the HKC2 from the coupling reaction.

FIG. 8. Shows the preparation of HKC2-PEG2k-RGD in two steps. The first step the coupling between c(RGDfk) and a bifunctional PEG molecule bearing N-hydroxySuccinimide (NHS) and Maleimide (Mal) functional groups, forming an amide bond through the coupling between amine and NHS. In the second step, the thiol in HKC (SEQ ID NO: 17) was reacted with maleimide of RGD-PEG2000-Mal to provide the RGD attached PEG linker polypeptide HKC2-PEG2000-RGD.

FIG. 9. Shows the characterization of RGD-PEG2k-Mal intermediate by ¹H NMR (DMSO-d6, 25° C.) spectroscopy. We can observe the RGD signals at 8.5-7.2 ppm and maleimide signal at 7.00 ppm, PEG ethylene broad peak at ˜3.5 ppm.

FIG. 10. Shows the stack plot comparison of the characterization of HKC2-PEG-RGD by ¹H NMR spectroscopy HKC2 derivatization with RGD targeting ligand.

FIG. 11. Shows the preparation of HKC1-PEGn-GalNAc (n=6, 12, 24) from the conjugation of HKC1 and trivalent GalNAc-PEG molecule bearing Maleimide (Mal) functional groups, forming a S-C bond through the coupling between thiol on cysteine and Mal. Figure discloses “KHHHKHHHKHHHKHHHKSSSC” as SEQ ID NO: 11.

FIG. 12. Formulation of HKC:HKP:TGFβ1 in the formation of nanoparticle and its size distribution. HKC=HKC2=K(HHHK)₄CSSC (SEQ ID NO: 1), HKP=H3K4b.

FIG. 13. Formulation of HKC:HKP:TGFβ1 in the formation of nanoparticle and its polydispersity index. HKC=HKC2=K(HHHK)₄CSSC (SEQ ID NO: 1), HKP=H3K4b.

FIG. 14. Effect of treatment with Cell Death siRNA formulated with HKP alone or in combination with various amount of HKP and HKC2 on human glioblastoma T98G cells viability. A aqueous solution of HKC2 (160 ng/μL), HKP (320 ng/μL) and siRNA (80 ng/μL) was mixed in the defined ratio and incubated at RT for 30 min. Transfection complexes were diluted with OPTI-MEM and added to the cells in 100 μL medium supplied with fresh medium. Transfection medium was replaced with 10% FBS/DMEM or EMEM in 6 h after. At 72 h post-transfection number of viable cells was assessed with CellTiter-Glo Luminescent cell viability assay (Promega). Values derived from untreated cells (Blank) were set as 100%. All values represent the mean of ±S.D. of four replicates NS-non-silencing siRNA, CD-Cell Death siRNA.

FIG. 15. Effect of treatment with Cell Death siRNA formulated with HKP alone or in combination with various amount of HKP and HKC2 on human hepatocellular carcinoma HepG2 cells viability. An aqueous solution of mixture of HKC2 (160 ng/μL), HKP (320 ng/μL) and siRNA (80 ng/μL) was incubated at RT for 30 min. Transfection complexes were diluted with OPTI-MEM and added to the cells in 100 μL medium supplied with fresh medium. Transfection medium was replaced with 10% FBS/DMEM or EMEM in 6 h after. At 72 h post-transfection number of viable cells was assessed with CellTiter-Glo Luminescent cell viability assay (Promega). Values derived from untreated cells (Blank) were set as 100%. All values represent the mean of ±S.D. of four replicates NS-non-silencing siRNA, CD-CellDeath siRNA.

FIG. 16. Formulation and nanoparticle formation through self-assembly among HKC2-PEG1k-folate, H3K4b (HKP) and siRNA. TGFβ1 was used in 80 ng/μL in water and mixed with equal volume of the HKC and HKP in water.

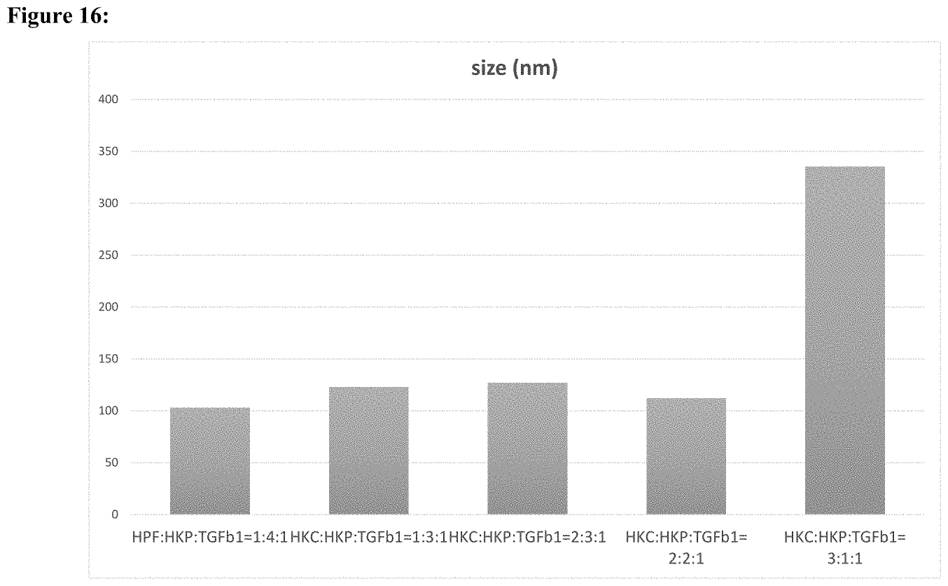

FIG. 17. Polydispersity index of nanoparticle formation through self-assembly among HKC1-PEG-folate, H3K4b (HKP) and siRNA. TGFβ1 (80 ng/μL in water) was mixed with equal volume of the HKC and HKP in water.

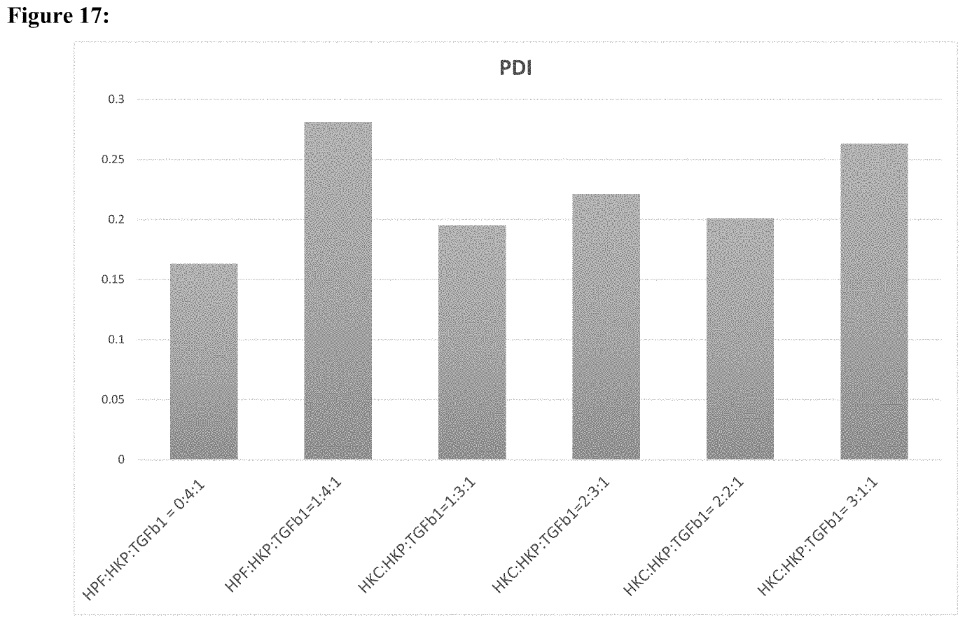

### DETAILED DESCRIPTION

Novel approaches are provided for tumor targeting nucleic acids for in vitro and in vivo delivery. As used herein, a histidine(H)-lysine(K) rich polypeptide (HKP) was used to describe the positively charged peptide with four branched repeating (H3K)4 units that contains a nucleic acid binding domain and provides non-cell specific transduction function (e.g. ability to cross cellular membranes non-selectively). Chou et al., Biomaterials, 35, 846-855 (2014). A linear peptide with a H3K four repeating units and a targeting ligand at the terminal site, (abbreviated as HKC), was used. This peptide comprises both a nucleic acid binding domain and cell-specific targeting function, so it can assist the material crossing the cellular membranes and specifically delivering the nucleic acid(s) into the specific cell type.

In some embodiments, compositions and methods are provided for delivering nucleic acid to target cells of interest. In some embodiments, the composition comprises a branched polypeptide (HKP) and linear peptide (HKC). In still another embodiment, this composition includes one or more nucleic acid. In some embodiments, the compositions include a pharmaceutically acceptable carrier.

In some embodiments, a four branched histidine-lysine rich polypeptide is used in the formulation with a linear peptide having certain structure and functional properties to be an effective carrier for: a) target nucleic acids to one or more particular cell types and b) delivery of the targeted nucleic acids to the particular intracellular location. In some embodiments, the linear peptide contains a cell specific targeting ligand (e.g., a small molecule, or cyclic peptide-based homing domain), which is conjugated with a positively charged linear HKC peptide that both binds to nucleic acid and provide the cell directing and transporting properties to help deliver the nucleic acid to the cytosol of the targeted cell.

In other aspects, methods are provided to conjugate a targeting ligand to the delivery carrier by a direct covalent linkage strategy. In some embodiments, the targeting ligand (e.g. folate, RGD or a peptide) was effectively conjugated with the linear histidine-lysine rich cysteine containing (HKC) peptide through chemical reaction by formation of a covalent bond. This method provides a versatile platform to introduce various targeting ligands to the delivery system for protecting the target nucleic acids. The chemical conjugation between the positive charged peptide HKC and the ligand can be disulfide bond, sulfur-carbon bond from thiol/maleimide, or any other covalent bonds or biodegradable bonds like hydrazine and amide, but not necessarily limited to this type.

In still other aspects, novel methods are provided for nanoparticle formulation of a polypeptide (HKP), a linear peptide bearing a targeting ligand, and an siRNA for tumor targeting. In some embodiments, the nucleic acid is delivered in complex that includes a targeting linear polypeptide comprising a motif that binds to a cellular target and a branched polypeptide. The two peptides were formulated in a defined ratio in a mixture with target nucleic acid in a nanoparticle formation. In some embodiments, the ratio of the negative charge (e.g. from the nucleic acid) to positive charge (e.g., in the peptide and polypeptide) of a peptide/nucleic acid complex can impact the strength of the non-cell-specific transduction properties of the complex.

In other aspects, compositions and methods are provided for delivering one or more nucleic acids to cellular targets. In some embodiments, in the peptide/nucleic acid complex, one or more nucleic acid was delivered in a nanoparticle at the same time. In some embodiments, the chemotherapy drug can be co-formulated within the nanoparticle complex. This provides the advantage and benefit in combinational therapy for treatment of the tumor.

Accordingly, these and other aspects provide a delivery platform that is a system into which can be introduced any type of targeting motif to target any cell of interest. In some embodiments, a stepwise method of conjugation a target ligand to peptide through a linker (e.g., peg or polymer) has been developed and presented in the application. The various targeting ligands provide specific transduction properties to any type of the cell of interest. In some embodiments, the binding domain in the peptide which has HK positive repeating units, it binds to the negative charged nucleic acid through hydrogen bond between the histidine and phosphate, and ion-ion interaction between the protonated lysine and phosphonate. The nucleic acid was protected and delivered to the targeted region of the cell of interest.

In terms of the targeting ligand, the peptide can be cyclic(c) RGD, APRPG (SEQ ID NO: 2), NGR, F3 peptide, CGKRK (SEQ ID NO: 3), LyP-1, iRGD, iNGR, T7 peptide (HAIYPRH (SEQ ID NO: 4)), MMP2-cleavable octapeptide (GPLGIAGQ (SEQ ID NO: 5)), CP15 (VHLGYAT (SEQ ID NO: 6)), FSH (FSH-β, 33-53 amino acids, YTRDLVKDPARPKIQKTCTF (SEQ ID NO: 7)), LHRH (QHTSYkcLRP (SEQ ID NO: 8)), gastrin-releasing peptides (GRPs) (CGGNHWAVGHLM (SEQ ID NO: 9)), RVG (YTWMPENPRPGTPCDIFTNSRGKRASNG (SEQ ID NO: 10)). In some embodiments, the targeting ligands can be incorporated into the bivalent or trivalent of homo- or hetero-peptide ligand combination in one system for better efficacy.

Accordingly, aspects of the invention provide a delivery platform that is modular and that can be adapted to deliver any nucleic acid to any cell of interest. In some embodiments, compositions are provided that contain multivalent peptide components and siRNA, mRNA, or DNA, and that forms a nanoparticle. The complex formation effectively protects and delivers the siRNA, mRNA, or DNA into the cell. In some embodiments, the nucleic acid is reversibly associated with the peptide carrier, which allows it to penetrate into the tumor specific cell and release the nucleic acid from the endosome to reach its target gene.

The production of an siRNA delivery carrier as described herein may occur by combining a branched polypeptide (HKP), linear peptide (HKC) and siRNA and may be implemented by a method that includes the steps of: (a) preparing the positive charged linear peptide e.g. peptide HKC having a functional group for linkage a targeting group or other functional moiety; (b) attaching the targeting ligand to the linear peptide HKC through a covalent bond and recovery of the product; (c) stably combining a branched polypeptide (HKP), a linear peptide HKC carrying a targeting ligand in step (b), and siRNA to produce homogeneous nanoparticles. In the above method, the steps may be also implemented at the same time, thereby allowing the preferable interaction and nanoparticle formation. The plyometric nanoparticle by this method effectively forms a composite with various siRNAs in aqueous solution to form polynanoparticles, which may be selectively accumulated in a specific disease via the targeting effect. Preferably, the size of the polynanoparticle as described herein may range from 10 nm to 3000 nm based on the described production method. Depending on the preclinical study, the preferred size would be in 40-300 nm as determined by dynamic light scattering.

In addition, the HKC polypeptide-nucleic acid delivery system described herein may be used as an effective ingredient of a pharmaceutical composition. Accordingly, pharmaceutical compositions are provided that contain a therapeutically effective dose in a mix form of HKC peptide and nucleic acid. It may include one or more kinds of the pharmaceutical compatible polymers or carriers in addition to the HKC polypeptide-nucleic acid delivery system as described herein, together with methods for their administration.

The resulting product may be formulated in forms such as powder, liquid, solid state, capsule, injectable, or the like, which may be mixed with one or more effective ingredients such as saline solution, buffer solution, or other compatible ingredients to maintain the stability and effectiveness of the nucleic acid-peptide polynanoparticle.

Pharmaceutical compositions as described herein may be administered by standard methods, including oral or parenteral administration.

### EXAMPLES

### Example 1. Synthesis of the Peptide HKC1 and HKC2

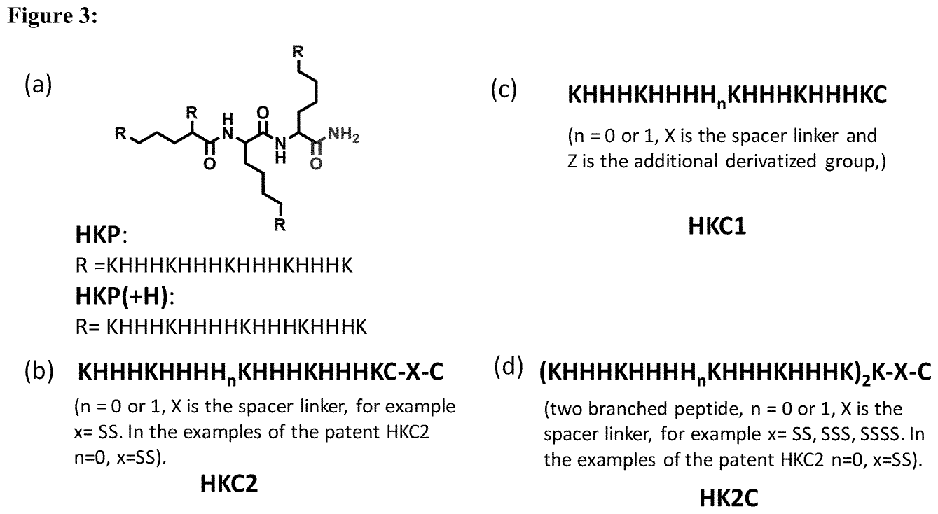

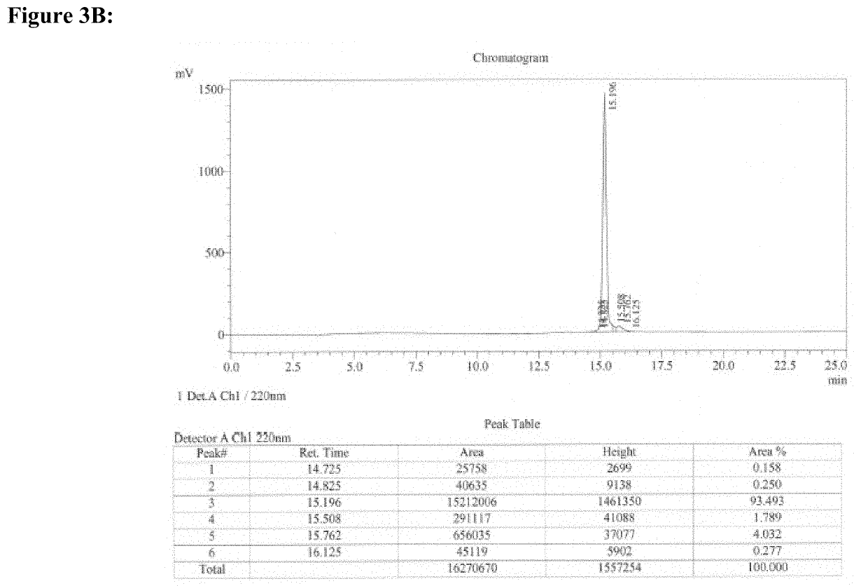

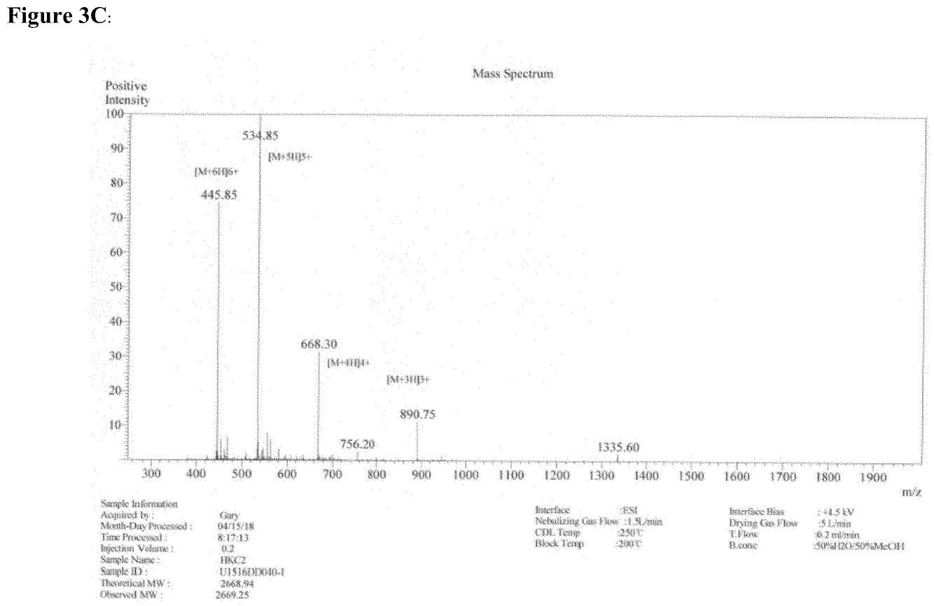

The designed peptide sequence of HKC1 (sequence: KHHHKHHHKHHHKHHHKSSSC (SEQ ID NO: 11)) was synthesized by solid state synthesizer as described in FIG. 3. The product was purified by the HPLC with water (0.065% TFA) and acetonitrile (0.05% TFA) and a chromatogram of HPLC in FIG. 3A. The structure of the H3K4C (in abbreviation HKC1) has one cysteine at the terminal site. The structure was further confirmed by the mass spectroscopy as shown in FIG. 3B.

The second designed peptide sequence of HK2C (sequence: (KHHHKHHHKHHHKHHH)₂KCSSC) was synthesized in a similar method by solid state synthesizer as shown in FIG. 3B.

The third designed peptide sequence of HKC2 (sequence: KHHHKHHHKHHHKHHHKCSSC (SEQ ID NO: 12)) was synthesized by solid state synthesis as described, for example, in U.S. Pat. Nos. 7,070,807, 7,163,695 and 7,772,201.

### Example 2. Cross-Linking of the HKC2 Peptide Via Sulfide Maleimide Coupling Reaction

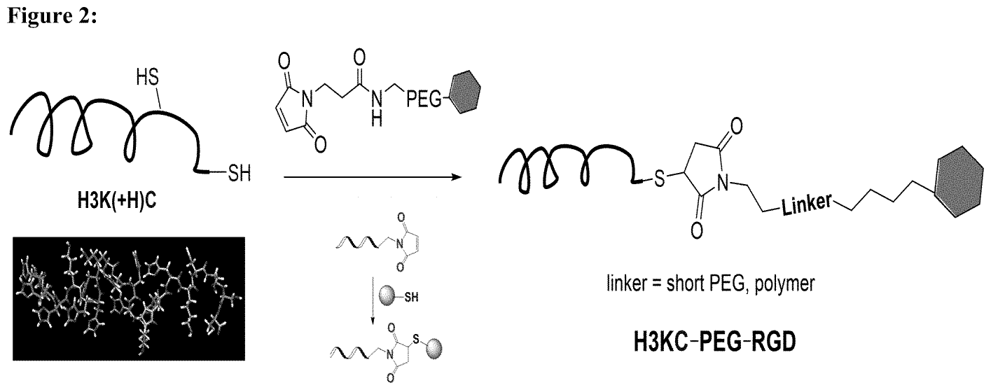

FIG. 2 shows the general scheme for coupling. For preparation of the H3K4C-PEG-targeting ligand functionalized polypeptide, functionalized PEG with a targeting motif is used. Such PEGs with targeting motifs such as folate, RGD, and/or monoclonal antibody, are commercially available or may be prepared in advance using methods that are well known in the art. HKC bearing a terminal cysteine was conjugated with a maleimide functionalized PEG linked targeting motif such as (folate, RGD, mAb, etc.) through thiol/maleimide addition reaction under mild conditions as shown in FIG. 2.

### Example 3. Cross-Linking of HKC2 Peptide with Folate

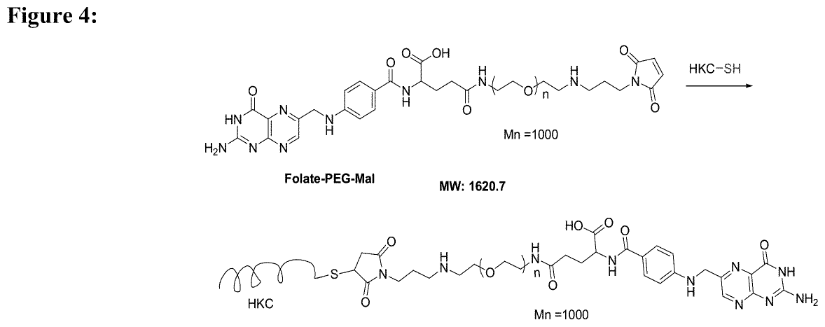

A targeting ligand was installed on the HKC peptide via formation of a covalent bond between the thiol and maleimide in a coupling reaction. FIG. 4 shows the scheme for preparing HKC2-PEG1000-folate. Folate-PEG1000-Mal (6.0 mg, 3.7 mmol) was dissolved in dry DMF (2.0 mL), followed by addition of trimethylamine (52 uL, 0.726 g/mL) in dry DMF. The HKC (10.0 mg, 3.7 mmol) in a mixture of degassed water (100 μL) and DMF (300 μL) by sonication and stirring was added to the mixture under the nitrogen at 25° C. The resulted mixture was stirred in the dark at 25° C. for 15 hours under nitrogen. HPLC analysis indicated that the starting material Folate-PEG1000-Mal was fully consumed and the reaction was completed. The reaction mixture was poured into to a cold diethyl ether solution to yield a yellow precipitate. The mixture was centrifuged at 4000 rpm for 10 min, and the top clear supernatant was discarded. The yellow precipitate was washed with acetone (5.0 mL) and centrifuged again to collect the product after discarding the supernatant. The product was further purified by preparative RP-HLPC or dialysis in water to obtain the pure product. The product solution was lyophilized to provide the product a yellow powder (12 mg, yield 75%).

### Example 4. Characterization of HKC2-PEG-Folate by ¹H NMR

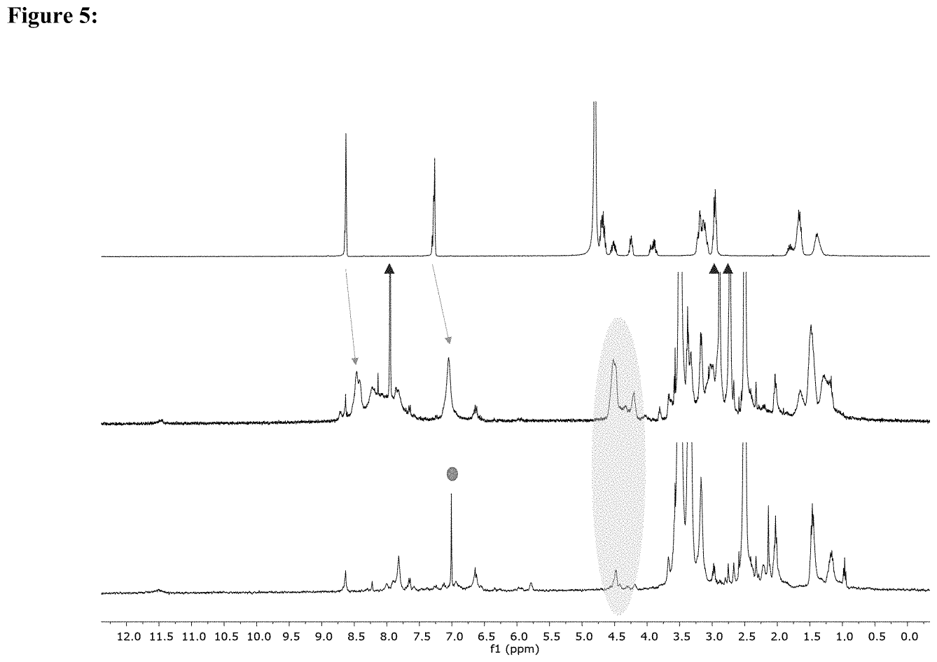

The structure of HKC2-PEG-folate was characterized by ¹H NMR in DMSO-d₆ and the results are shown in FIG. 5. The sample of ˜5 mg was dissolved in D₂O or DMSO-d₆ and the nmr spectra were recorded at 400 MHz. The three spectra were superimposed to clearly see the difference. HKC2 in D₂O was on the top, HKC2-PEG-folate in DMSO-d₆ was at the middle, and folate-Peg1000-Mal was presented at the bottom. The HKC was covalently coupled with folate-Peg1000-Mal, and the characteristic signal of maleimide double bond at 7.0 ppm disappeared after reaction with cysteine. The CH₂ proton of PEG group is present at the region 3.5 ppm and peptide protons are located at 6.0-9.0 ppm in the HKC2-PEG-folate.

### Example 5. Characterization of HKC2-PEG-Folate by UV/Vis Spectroscopy

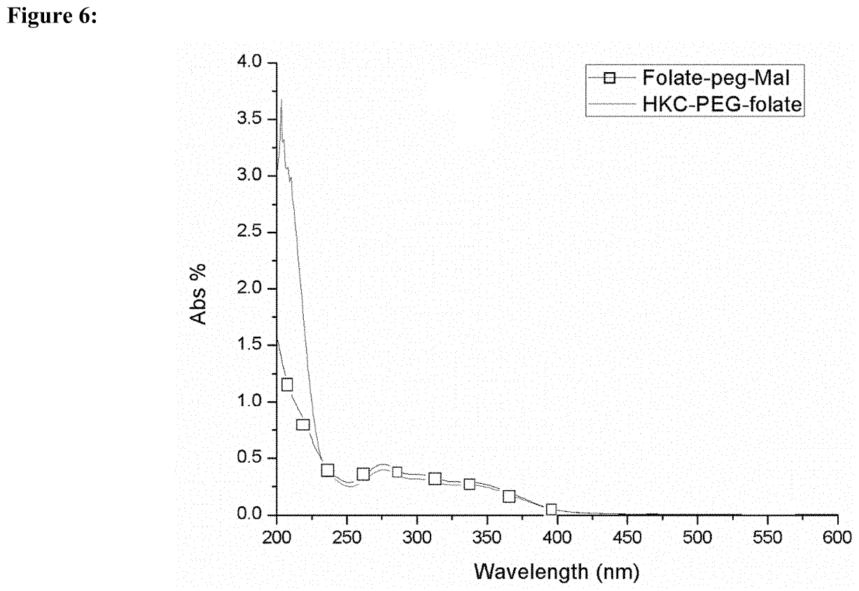

The structure of HKC2-PEG-folate was further characterized by UV/Vis spectroscopy and the result is shown in FIG. 6. The UV/Vis spectroscopy of HKC2-PEG-folate (top red curve) and Folate-PEG-Mal (bottom gray curve) were measured in water at room temperature. The characteristic absorbance at the 220 nm for the peptide and 275 nm for the folate was observed in the product spectrum.

### Example 6. Characterization of HKC2-PEG-Folate by Mass Spectroscopy

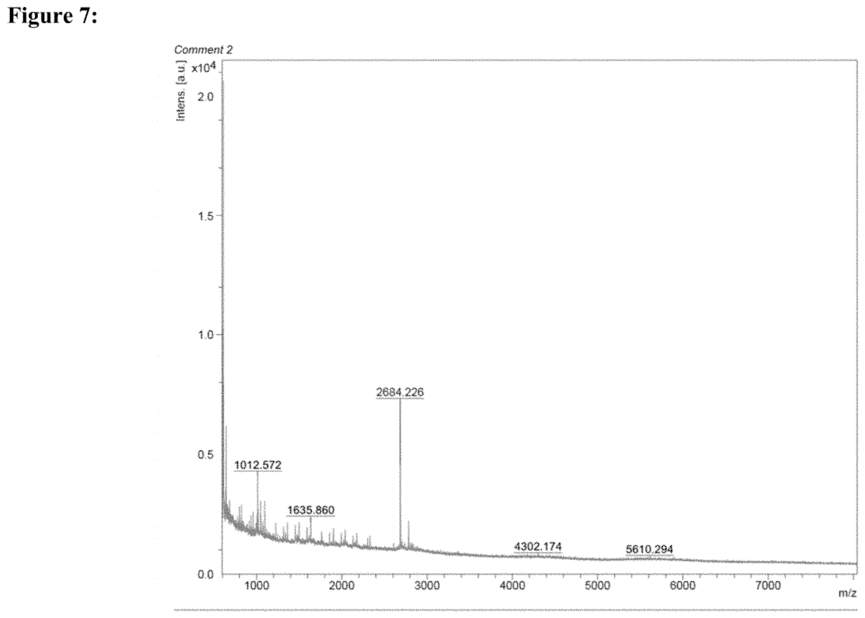

MALDI-MS (positive) spectroscopy of the HKC2-PEG-folate was recorded using a Bruker Autoflex Speed spectrometer. Presence of the molecular ion peak around 4302 M⁺ indicates successful conversion of the HKC1 from the coupling reaction. See FIG. 7.

### Example 7. Preparation of HKC2 Containing RGD Ligand as HKC2-PEG2k-RGD

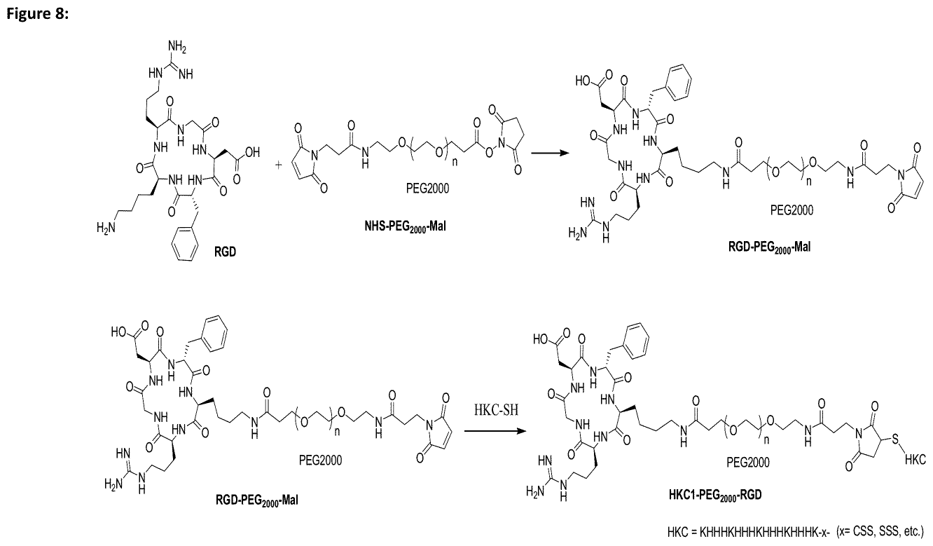

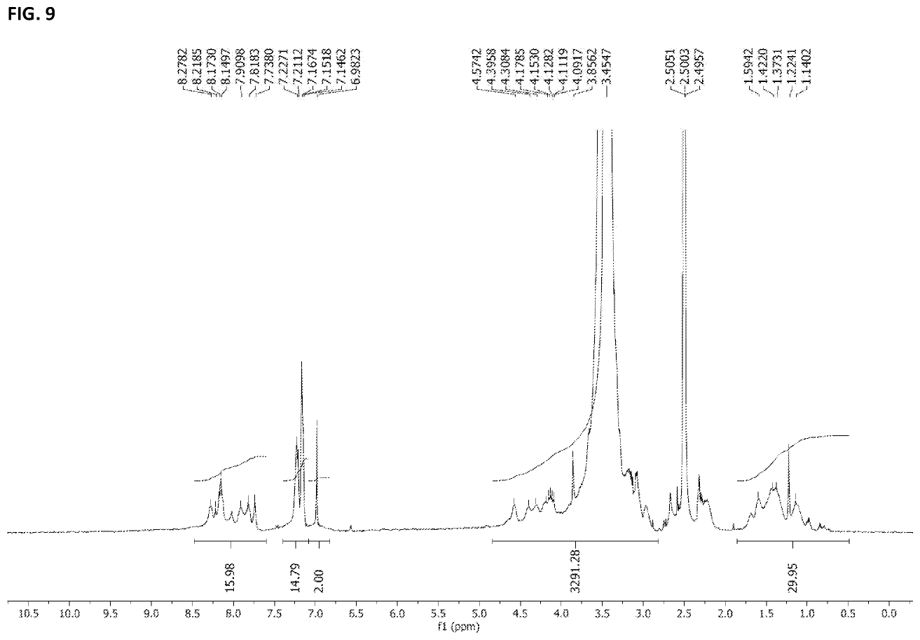

First step: coupling between c(RGDfk) and a bifunctional PEG molecule bearing N-hydroxy Succinimide (NHS) and Maleimide (Mal) functional groups, forming a amide bond via coupling between the amine and NHS ester. See FIG. 8. c(RGDfk) (5.0 mg, 8.28 μmol) was dissolved in dry DMF (1 mL), and triethylamine (10 μL) was added. After the resulted mixture was stirred for 30 min at room temperature under the N₂, the Mal-PEG2k-NHS (10 mg, 8.28 μmol) was added in one portion and stirred for 12 hours at 25° C. The reaction mixture was poured into a cold diethyl ether (20 mL). The mixture was centrifuged at 4000 rpm for 10 min at 5° C., and the top clear supernatant was discarded. The white precipitate was resuspended in acetone and cold diethyl ether (10 mL) was added and sonicated for 5 min. Centrifugation again at 4000 rpm for 10 min at 5° C. allowed collection of the white precipitate. Drying under vacuum afforded RGD-PEG2k-Mal (12 mg, 80% yield). The ¹H NMR spectrum (400 MHz, DMSO-d₆), showed presence of a peak assigned to the maleimide at 7.0 ppm, but absence of the peak for the NHS ester (2.8 ppm). See FIG. 9.

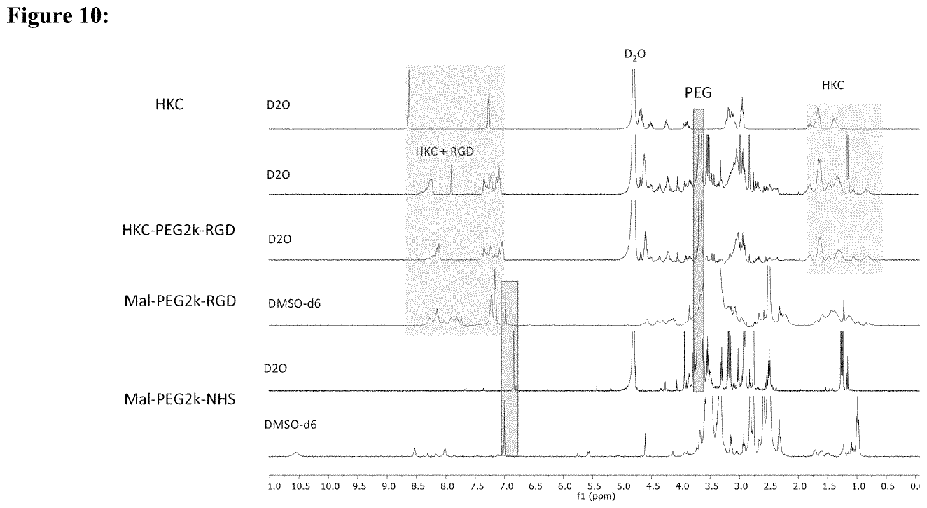

This material was used directly in the second step, where the thiol in HKC reacted with the maleimide of RGD-PEG2k-Mal to provide the RGD attached PEG linker polypeptide HKC2-PEG2k-RGD. HKC2 (5.4 mg, 2.0 μmol) was dissolved in a mixture of DMF (0.6 mL) and degassed water (100 μL). The solution of HKC2 was added to the RGD-PEG2k-Mal (5.0 mg, 1.69 μmol) dissolved in dry DMF (1 mL) under stirring. Triethylamine (100 uL, 10 μg/μL in dry DMF) was subsequently added and the mixture was stirred at 25° C. for 15 hours under N₂. The reaction mixture was poured into cold diethyl ether (20 mL). The mixture was centrifuged at 4000 rpm for 10 min at 5° C., and the top clear supernatant was discarded. The crude product was dialyzed against water for 2 days with changes of water. After drying under vacuum, the product HKC2-PEG2k-RGD was afforded (7.1 mg, 75% yield). The product was characterized by spectroscopy method including ¹H NMR (see FIG. 10) and mass spectrometry.

### Example 8. Preparation of HKC Containing Trivalent GalNAc Ligand as HKC-PEGn-GalNAc

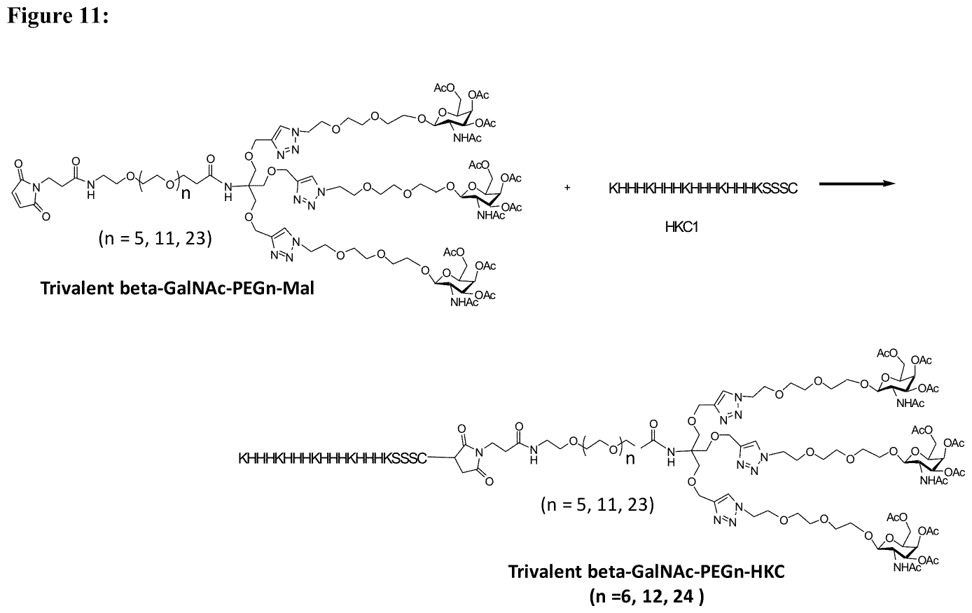

HKC1 ((KHHH)4KSSC (SEQ ID NO: 13)), 18.0 mg, 6.75 μmol) was dissolved in pH=7.2 phosphate buffer in a glass vial. The GalNAc3-PEG6-Mal (29.3 mg, 1.56 μmol) in dry DMF (300 μL) was added by springe needle to the HKC1 solution over 5 min. The resulting mixture was stirred under a nitrogen atmosphere for 16 hours. After HPLC monitoring showed that the starting material GalNAc was fully consumed, the crude product was purified using a Pierce Dextran desalting column to result the pure product GalNAc3-PEG6-HKC1 as a white solid in 19 mg, 80% yield. The product was characterized by mass spectrometry as (MALDI-TOF-MS positive) m/z 4595.824 [M+H], Calculated MW=4595.9. HPLC analysis showed purity >90%. GalNAc-PEG12-HKC1 and GalNAc-PEG24-HKC1 were prepared in a similar method by replacing the GalNAc3-PEG6-Mal with the corresponding GalNAc-PEG12-Mal and GalNAc-PEG24-Mal. (reaction scheme shown in FIG. 11).

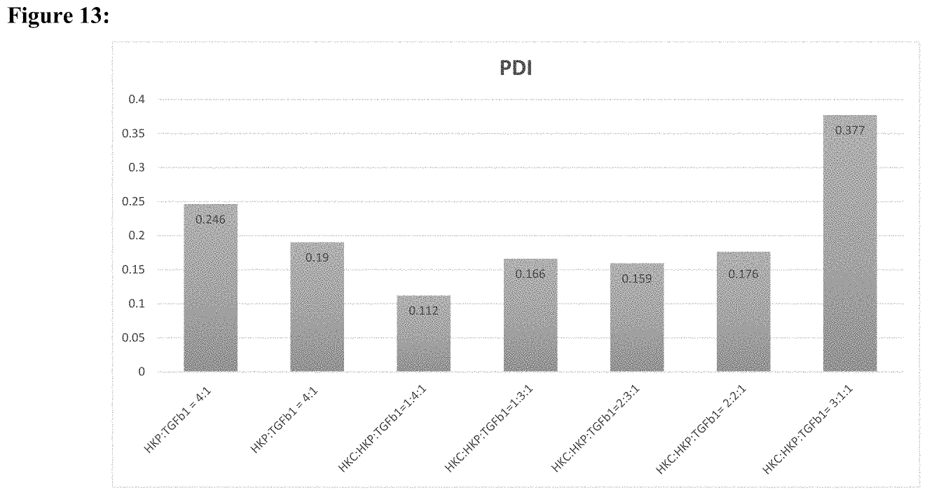

### Example 9. Formulation of HKC:HKP:TGFβ1 in the Formation of Nanoparticle and its Size Distribution. HKC=HKC2=K(HHHK)₄CSSC (SEQ ID NO: 1). HKP=H3K4b. (FIG. 13)

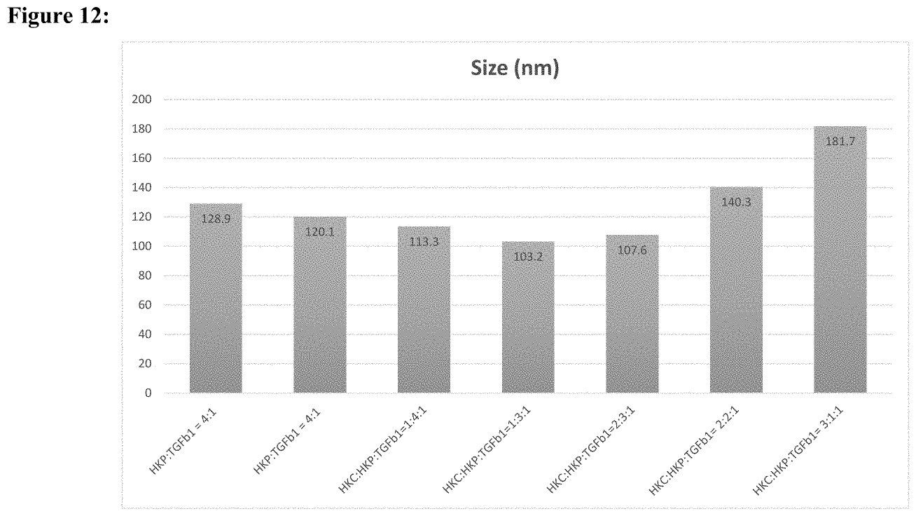

The nanoparticle formation of HKC2, HKP and siRNA (TGFβ1) was evaluated in various ratios. Addition of HKC2 into the HKP/siRNA formulation maintained the similar nanoparticle size but significantly narrowed the polydispersity index (PDI) as compared to the control HKP/siRNA (N:P mass ratio=4:1). The HKC2/HKP/siRNA was formulated in mass ratio 0:4:1, 1:4:1, 1:3:1, 2:3:1, 2:2:1, 3:1:1. An aqueous solution of HKC2 (160 ng/μL), HKP (320 ng/μL) and siRNA (80 ng/μL) was mixed in the defined ratio and incubated at RT for 30 min. The resulted sample was subsequently measured by dynamic light scattering using a Nanoplus 90. The dynamic radius and the polydispersity index were recorded and are shown in FIGS. 11 and 12. From the FIG. 12, the size was reduced slightly from 120 nm (HKP:siRNA=4:1) to 100-113 nm (HKC2/HKP/siRNA=1:4:1, 1:3:1, 2:3:1). When the ratio increased to 2:2:1, 3:1:1, the nanoparticle size also increased to 140 and 180 nm. In another point of view, the PDI was reduced from 0.22 to 0.11-0.17, which is the benefit of the HKCs filling up the covered surface (see FIG. 13).

### Example 10. Effect of Treatment with Cell Death siRNA Formulated with HKP Alone or in Combination with Various Amount of HKP and HKC2 on Human Glioblastoma T98G Cells Viability

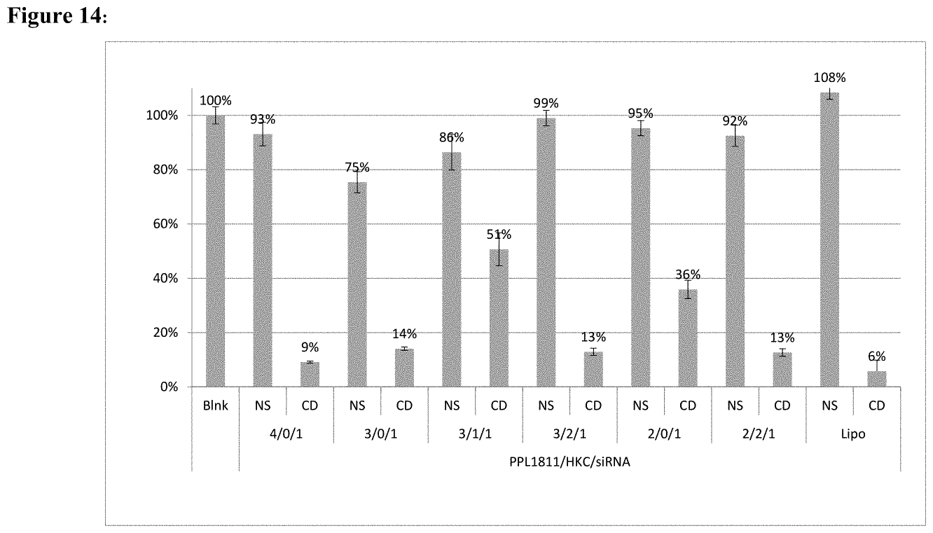

An aqueous solution of HKC2 (160 ng/μL), HKP (320 ng/μL) and siRNA (80 ng/μL) was mixed in the defined ratio (HKC2/HKP/siRNA was formulated in mass ratio 0:4:1, 0:3:1, 1:3:1, 2:3:1, 0:2:1, 2:2:1) and incubated at RT for 30 min. Transfection complexes were diluted with OPTI-MEM and added to the cells in 100 μL medium supplied with fresh medium. Transfection medium was replaced with 10% FBS/DMEM or EMEM after 6 h. At 72 h post-transfection the number of viable cells was assessed with a CellTiter-Glo Luminescent cell viability assay (Promega). Values derived from untreated cells (Blank) were set as 100%. All values represent the mean of ±S.D. of four replicates NS-non-silencing siRNA, CD-CellDeath siRNA. Lipofectamine and HKP/siRNA (4:1) were used as the positive control. The addition of HKC2 in the formulation of 2:3:1 and 2:2:1 showed comparable or even better cell death in terms of cell viability comparing to the control 0:3:1 and 0:2:1 (FIG. 14).

### Example 11. Effect of Treatment with Cell Death siRNA Formulated with HKP Alone or in Combination with Various Amounts of HKP and HKC2 on Human Hepatocellular Carcinoma HepG2 Cells Viability

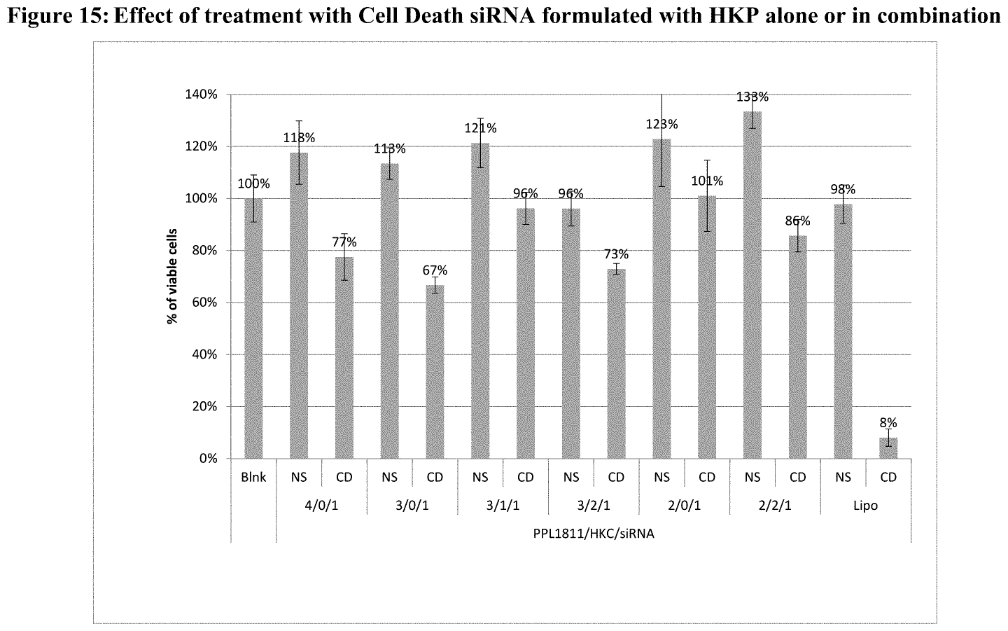

An aqueous solution of a mixture of HKC2 (160 ng/μL), HKP (320 ng/μL) and siRNA (80 ng/μL) was mixed in a defined ratio (HKC2/HKP/siRNA was formulated in mass ratio 0:4:1, 0:3:1, 1:3:1, 2:3:1, 0:2:1, 2:2:1) and incubated at RT for 30 min. Transfection complexes were diluted with OPTI-MEM and added to the cells in 100 μL medium supplied with fresh medium. Transfection medium was replaced with 10% FBS/DMEM or EMEM after 6 h. At 72 h post-transfection the number of viable cells was assessed with CellTiter-Glo Luminescent cell viability assay (Promega). Values derived from untreated cells (Blank) were set as 100%. All values represent the mean of ±S.D. of four replicates NS-non-silencing siRNA, CD-CellDeath siRNA. Lipofectamine and HKP/siRNA (4:1) were used as the positive control (FIG. 15). The addition of HKC2 in the formulation of 2:3:1 and 2:2:1 showed comparable or even better cell death percentage in terms of the cell viability comparing to the control 0:3:1 and 0:2:1, although the overall cell viability is higher than the human glioblastoma T98G cell line study.

All publications identified herein, including issued patents and published patent applications, and all database entries identified by url addresses or accession numbers are incorporated herein by reference in their entireties.

Although this invention has been described in relation to certain embodiments thereof, and many details have been set forth for purposes of illustration, it will be apparent to those skilled in the art that the invention is susceptible to additional embodiments and that certain of the details described herein may be varied without departing from the basic principles of the invention.

1. A linear or branched peptide comprising a nucleic acid binding domain and a cell-specific targeting ligand, wherein said nucleic acid binding domain comprises the sequence K(HHHK)₄XC (SEQ ID NO: 14), wherein ‘X’ is a linker between the terminal cysteine and said binding domain.

2-7. (canceled)

8. The peptide of claim 1, wherein the peptide is selected from the group consisting of HKC1, HKC2, and HK2C.

9-11. (canceled)

12. The peptide of claim, wherein the linker comprises a polymeric spacer molecule.

13. (canceled)

14. The peptide of claim 1, wherein the cell specific targeting ligand is selected from the group consisting of a small molecule, a peptide, a protein, an antibody, and an aptamer.

15. (canceled)

16. The peptide of claim 14, wherein the number of targeting ligands is 1-4.

17. The peptide of claim 14, wherein the targeting peptide is selected from the group consisting of cyclic(c) RGD, APRPG (SEQ ID NO: 2), NGR, F3 peptide, CGKRK (SEQ ID NO: 3), LyP-1, iRGD, iNGR, T7 peptide (HAIYPRH (SEQ ID NO: 4)), MMP2-cleavable octapeptide (GPLGIAGQ (SEQ ID NO: 5)), CP15 (VHLGYAT (SEQ ID NO: 6)), FSH (FSH-β,33-53 amino acids), YTRDLVKDPARPKIQKTCTF (SEQ ID NO: 7)), LHRH (QHTSYkcLRP (SEQ ID NO: 8)), gastrin-releasing peptides (GRPs) (CGGNHWAVGHLM (SEQ ID NO: 9)), and RVG (YTWMPENPRPGTPCDIFTNSRGKRASNG (SEQ ID NO: 10)).

18. A composition comprising the peptide of claim 1 and a branched polypeptide with histidine (H) and lysine (K) rich repeating units, wherein the branched polypeptide comprises four branches of K(HHHK)₄ (SEQ ID NO: 14), or KHHHKHHHHKHHHKHHHK-repeating units (SEQ ID NO: 18).

19-22. (canceled)

23. A composition comprising the peptide of claim 1 and a nucleic acid.

24. The composition of claim 23, wherein the nucleic acid is selected from the group consisting of an siRNA, an miRNA, an antisense oligonucleotide, a plasmid, an mRNA, an RNAzyme, a DNAzyme, and an aptamer sequence.

25-28. (canceled)

29. The composition of claim 23 further comprising a second nucleic acid.

30. The composition of claim 29, wherein the first nucleic acid sequence is an siRNA and the second nucleic acid is an siRNA, an miRNA, an antisense oligo, a plasmid, an mRNA, an RNAzyme, a DNAzyme, or an aptamer sequence.

31-37. (canceled)

38. A method of delivering a nucleic acid to a mammalian cell comprising contacting the cell with the composition of claim 23.

39. The method of claim 38, wherein the nucleic acid is delivered to the cell in vitro.

40. The method of claim 38, wherein the nucleic acid is delivered to the cell in vivo.

41-42. (canceled)

43. The method of claim 38, wherein the mammalian cell is a human cell.

44. A method of treating a mammal comprising administering a therapeutically effective amount of the composition of claim 23 to the mammal.

45-46. (canceled)

47. The method of claim 44, wherein the mammal is a human.

48-52. (canceled)

53. A method of preparing the composition of claim 23 comprising the steps of: a) mixing the peptide of claim 1 with a nucleic acid to form a complex, b) adding a branched polypeptide with histidine (H) and lysine (K) rich repeating units, wherein the branched polypeptide comprises four branches of K(HHHK)4 (SEQ ID NO: 14), or KHHHKHHHHKHHHKHHHK-repeating units (SEQ ID NO: 18) at a defined ratio to the mixture to form a nanoparticle, and b) recovering the nanoparticle.

54-60. (canceled)

61. The method of claim 53, wherein the nanoparticle size is 50-300 nm.

62. The method of claim 53, wherein the nucleic acid is an siRNA, an miRNA, an antisense oligo, a plasmid, an mRNA, an RNAzyme, a DNAzyme, or an aptamer sequence.

63-64. (canceled)
